(19) United States
(12) Patent Application Publication
(10) Pub. No.: US 2006/0105398 A1
(43) Pub. Date: May 18, 2006

(54) Methods of identifying compounds that modulate igg mediated mast cell activation

(75) Inventor: Alexander B. Rossi, Reedsport, OR (US)

Correspondence Address: DECHERT LLP, P.O. BOX 10004, PALO ALTO, CA, 94303, US

(73) Assignee: Rigel Pharmaceuticals, Inc, South San Francisco, CA (US)

(21) Appl. No.: 10/524,233

(22) PCT Filed: Aug. 6, 2003
(86) PCT No.: PCT/US03/24650

§ 371 Date: Aug. 29, 2005

Related U.S. Application Data
(60) Provisional application No. 60/402,288, filed on Aug. 9, 2002.

(51) Int. Cl. G01N 33/567; G01N 33/53

(52) U.S. Cl. 435/7.2

(57) ABSTRACT

The present invention relates to methods for identifying compounds capable of modulating Fcg receptor signaling pathway in mast and/or basophil cells. The invention further relates to methods for identifying compounds for use as therapeutic agents in the treatment of disorders related to activation of Fcg receptor signaling cascade.

### CROSS REFERENCES TO RELATED APPLICATIONS

This application claims the benefit of U.S. Provisional Application No. 60/402,288, filed Aug. 9, 2002, which is incorporated herein by reference.

### FIELD OF THE INVENTION

The present invention relates to methods of identifying compounds capable of modulating Fcγ receptor-mediated signaling cascade in mast and basophil cells and identifying compounds for treating disorders related to activation via the Fcγ signal transduction pathway. The present invention further relates to methods of identifying compounds capable of modulating IgE-dependent enhancement of Fcγ receptor-mediated mast and/or basophil cell activation.

### BACKGROUND OF THE INVENTION

Crosslinking of Fc receptors, such as the high affinity receptor for IgE (FcεRI) and/or the high affinity receptor for IgG (FcγRI) activates a signaling cascade in mast, basophil and other immune cells that results in the release of chemical mediators responsible for numerous adverse events. In mast and basophil cells, activation of the FcεRI or FcγRI signaling cascade leads to the immediate (ie., within 1-3 min. of receptor activation) release of preformed mediators of atopic and/or Type I hypersensitivity reactions (e.g., histamine, proteases such as tryptase, etc.) via the degranulation process. Such atopic or Type I hypersensitivity reactions include anaphylactic reactions to environmental and other allergens (e.g., pollens, insect and/or animal venoms, foods, drugs, contrast dyes, etc.), anaphylactoid reactions, hay fever, allergic conjunctivitis, allergic rhinitis, allergic asthma, atopic dermatitis, eczema, urticaria, mucosal disorders, tissue disorders and certain gastrointestinal disorders.

The immediate release of the preformed mediators via degranulation is followed by the release and/or synthesis of a variety of other chemical mediators, including, among other things, platelet activating factor (PAF), prostaglandins and leukotrienes (e.g., LTC4) and the de novo synthesis and release of cytokines such as TNFα, IL-4, IL-5, IL-6, IL-13, etc. These “late stage” mediators are thought to be in part responsible for the chronic symptoms of atopic and Type I hypersensitivity reactions, and in addition are chemical mediators of inflammation and inflammatory diseases (e.g., osteoarthritis, inflammatory bowel disease, ulcerative colitis, Crohn's disease, idiopathic inflammatory bowel disease, irritable bowel syndrome, spastic colon, etc.), low grade scarring (e.g., scleroderma, increased fibrosis, keloids, post-surgical scars, pulmonary fibrosis, vascular spasms, migraine, reperfusion injury and post myocardial infarction), and sicca complex or syndrome.

Generally, the major pathway of mast and basophil cell activation, and pathogenesis of allergic diseases, is believed to occur through IgE-associated signal transduction via FcεRI receptors. Contact with a variety of allergens is correlated with elevated levels of allergen specific IgE antibodies, and mice with targeted gene disruption of FcεRI α or γ chain display deficits in anaphylactic responses (Galli, S. J. and Lantz, C. S., Allergy, in Fundamental Immunology, 4th Ed., Paul W. E. ed., Lippincott-Raven Publishers, New York (1994)). Moreover, crosslinking of FcεRI by IgE and multivalent antigen or anti-IgE antibodies produces robust degranulation of mast cells and subsequent synthesis of late stage mediators.

The presence of various Fcγ receptors on mast cells and basophil cells, however, suggests that IgG-dependent activation also participates in allergic responses of these cells and other host defense mechanisms. For example, targeted gene disruption of Fcε receptor does not completely eliminate anaphylactic responses to allergens, and mast cells degranulate upon IgG-mediated activation of Fcγ receptor signaling cascade (Okayama, Y. et al. J. Immunol. 164(8):4332-4339 (2000)). The response of mast cells to Fcγ receptor activation, however, is lower than those seen for FcεR activation. Resting human mast cells express low levels of high affinity FcγR1, and subjecting the cells to receptor crosslinking conditions does not result in detectable levels of histamine release (Okayama et al., supra). In mouse mast cells known to express FcγRII and FcγRIII receptors, activation of these Fcγ receptors results in release of granule-associated mediators and enzymes, but at levels below that observed for FcεRI receptor activation (Katz, H. R. et al., J. Immunol. 148(3):868-871 (1992)). The decreased responsiveness of mast cells to Fcγ receptor activation presents a disadvantage in screens for compounds that modulate Fcγ receptor-mediated signaling, and consequently the identification of compounds with therapeutic applications, particularly those that may distinguish between IgG and IgE-mediated cellular responses. It is known that interferon gamma (IFN-γ) upregulates FcγRI in mast cells, thus providing a method of enhancing signal transduction by FcγRI (Okayama et al., supra). IFN-γ, however, potentially affects other cellular regulatory mechanisms that may lead to changes in the phenotypic character of mast cell populations used in a screen. The possible pleiotropic effects of exposing mast or basophil cells to IFN-γ complicate screens for compounds that modulate Fcγ signaling pathways. Thus, it is desirable in the art to have additional ways of examining Fcγ receptor-mediated signaling in mast and/or basophil cells, in addition to methods using FcγRI upregulation by IFN-γ.

### SUMMARY OF THE INVENTION

The present invention relates to methods of identifying compounds that modulate Fcγ receptor-mediated signaling pathways. The methods take advantage of the ability of IgE antibody to enhance Fcγ dependent activation of mast cells. These IgE primed mast cells display increased degranulation to Fcγ receptor activation, thereby providing mast cells useful in identifying compounds that modulate Fcγ mediated signaling pathway. It is to be understood that the methods described herein also apply to basophil cells as well.

Accordingly, in one aspect, the present invention provides a method of identifying compounds that are capable of modulating Fcγ receptor-mediated signaling in mast and/or basophil cells. The method generally comprises contacting at least one IgE primed mast or basophil cell with a candidate compound in the presence of Fcγ signaling cascade activation. The primed cells are made by contacting the cells with IgE antibody, which may be naturally occurring or recombinant IgE; monoclonal or polyclonal; or whole IgE molecule or IgE fragments containing the Fc region. The cell is then evaluated to determine whether the compound modulates Fcγ receptor-mediated mast and/or basophil cell activation.

The methods of the present invention may be used to identify compounds with therapeutic potential for treating or preventing diseases related to Fcγ receptor-mediated mast and/or basophil cell activation. Diseases associated with mediator release include, by way of example and not limitation, atopy or anaphylactic hypersensitivity or allergic reactions, allergies, low grade scarring, diseases related to tissue destruction, often associated with inflammatory reactions.

In a further aspect, the present invention provides for a method of identifying a compound capable of modulating IgE-mediated priming of mast and/or basophil cells. The method generally comprises contacting at least one mast or basophil cell with a candidate compound and priming the cell with IgE antibody. The treated cells are activated by stimulating Fcγ receptor signaling cascade to determine whether the candidate compound modulates IgE priming of the mast or basophil cells. Preferably, the cells are contacted with the candidate compound prior to IgE priming to distinguish effects of the compound on cell priming as compared to effects on Fcγ receptor-mediated cell activation.

The cells for screening may be isolated from tissues or comprise cultured cells. Preferably for screening purposes, cultured mast or basophil cells are used because of the ability to generate large numbers of cells, particularly by culturing the mast and basophil cells from hematopoietic stem cells. Screened mast cells may be of a specific phenotype, such as mucosal mast cells and connective tissue-type mast cells. In humans, musocal mast cells are typically tryptase-positive/chymase negative while connective tissue-type mast cells are typically tryptase and chymase positive. Any animal can serve as sources for mast and/or basophil cells, including mice and rats and other commonly used experimental animals, to higher animals, such as primates, particularly human subjects.

The candidate compound can be any compound, including but not limited to, small organic molecules, saccharides, carbohydrates, polysaccharides, lectins, peptides, proteins, nucleic acids, and the like. Preferably, the candidate compounds comprise small organic molecules with a molecular weight of about 100-2500 daltons. The small organic molecules may comprise cyclic structures of carbon atoms, or combination of carbon atoms and heteroatoms, and/or aromatic, polyaromatic, and heteroaromatic structures.

For embodiments of the method using candidate compounds capable of traversing the cell membrane, the candidate compounds may be administered to the cell by contacting the cell with the compounds. In embodiments of the methods in which the candidate compounds can be expressed or transcribed, such as proteins or RNA, the candidate compounds are administered to the cell as polynucleotides capable of expressing or transcribing the compound in the cell.

The methods of the present invention provide for contacting the mast and/or basophil cells with candidate compounds in the presence of Fcγ receptor signaling activation. Three types of Fcγ receptors are known, including FcγRI, FcγRII, and FcγRIII, and each may be subject to activation in the present invention. In one aspect, a specific Fcγ receptor signaling pathway is selectively activated. Preferential activation of one receptor pathway is done by contacting the mast and/or basophil cells with antibodies, either whole antibodies or F(ab)₂ fragements, directed against the specific receptor, and crosslinking of the receptor bound antibody or F(ab)₂ fragment. In one embodiment, the FcγRI receptor is selectively activated.

In the present invention, determining whether a compound is capable of modulating Fcγ receptor-mediated mast cell activation is done by evaluating cellular responses downstream of Fcγ receptor activation. These include determining release of preformed mediators by mast or basophil cell degranulation, or determing synthesis of late phase mediators.

In a final aspect, the present invention provides for kits for carrying out the methods of the invention. In one embodiment, the kit comprises mast and/or basophil cells, IgE antibodies for priming of the cells, components for activating the cells via the Fcγ receptor signaling pathway, and assay reagents for determining level of cell activation. Additional components may accompany the kits, including, by way of example and not limitation, buffers, labels, enzyme substrates, culture medium, and instructions describing use of the kits.

### BRIEF DESCRIPTION OF THE FIGURES

FIGS. 1A and 1B show the analysis of tryptase activity in the supernatant for stimulated populations of IgE primed mast cells at various IgG concentrations (human IgG3) and various dilutions of rabbit anti-human IgG. Five concentrations of IgG were tested at six different dilutions of anti-IgG antibody in the absence (FIG. 1A) or presence (FIG. 1B) of IL-4 in the priming solution. Controls included activation with ionomycin or stimulation with media lacking antibody. Addition of IL-4 to the priming solution did not appear to substantially affect activation;

FIGS. 2A and 2B show analysis of tryptase activity in the supernatant of stimulated populations of IgE primed mast cells and at various concentrations of IgG (human IgG3) and activated using various dilutions of rabbit anti-human IgG. Cells were stimulated with either anti-IgG alone (FIG. 2A) or in conjunction with anti-IgE (rabbit anti-human IgE) antibody (FIG. 2B);

FIG. 3 shows plots of FACS-sorted cells immunostained for various Fcγ receptor types without priming (SCF/IL-6) or with priming (SCF/IL-6/IL-4/IgE). Plots left of the vertical line show staining with antibodies directed to three different Fcγ receptor types, including FcγRI. Plots right of the vertical line show staining with antibodies against human IgE and IgG following incubation with either IgE or various types of IgG. IgG4 is known to bind only FcγRI. The data shows that levels of FcγRI increase in cells primed with IgE;

FIG. 4 shows plots of FACS sorted mast cells immunostained with anti-IgE/G under various priming conditions and incubated with either IgE or various types of IgG;

FIGS. 5A-5C show the effects of increasing concentrations of five different compounds which inhibit degranulation, as measured by tryptase release into the media by mast cells following activation with anti-IgE (FIG. 5A), anti-IgG (FIG. 5C), or both anti-IgE/G (FIG. 5B);

FIG. 6A-6E show that each of the inhibitory compounds A-E inhibits degranulation similarly, regardless of the mode of activation;

FIG. 7 shows tryptase activity in supernatants of cultured mast cells activated under various conditions. Culture conditions are shown on the right side of the figure. Activation conditions are indicated on the lower portion of each bar graph. In the presence of priming with IgE, mast cells show higher levels of tryptase activity upon activation of Fcγ signaling pathway with IgG4 and anti-IgG.

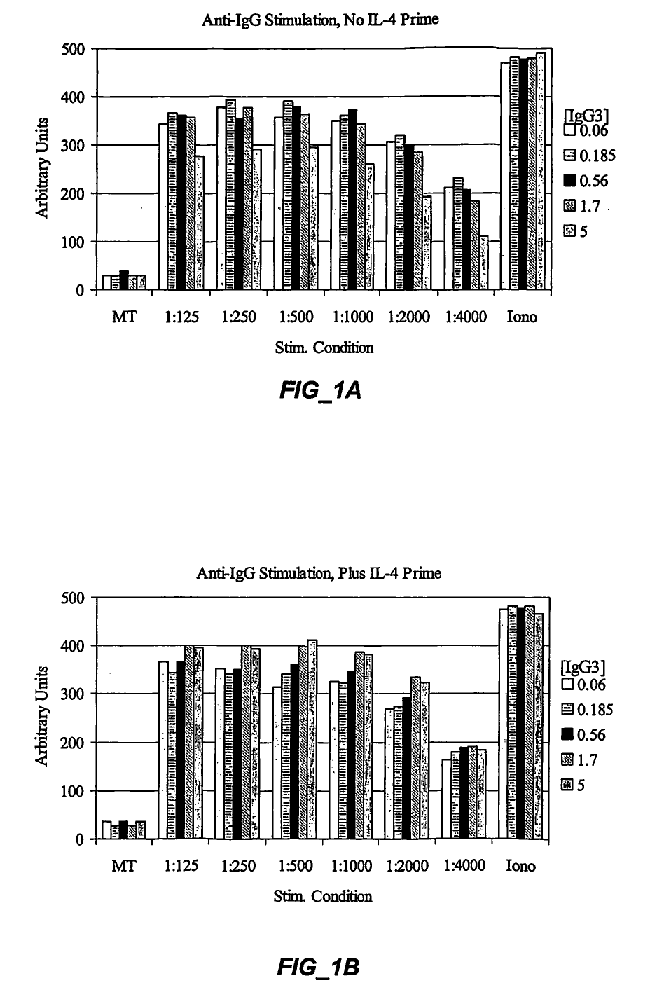

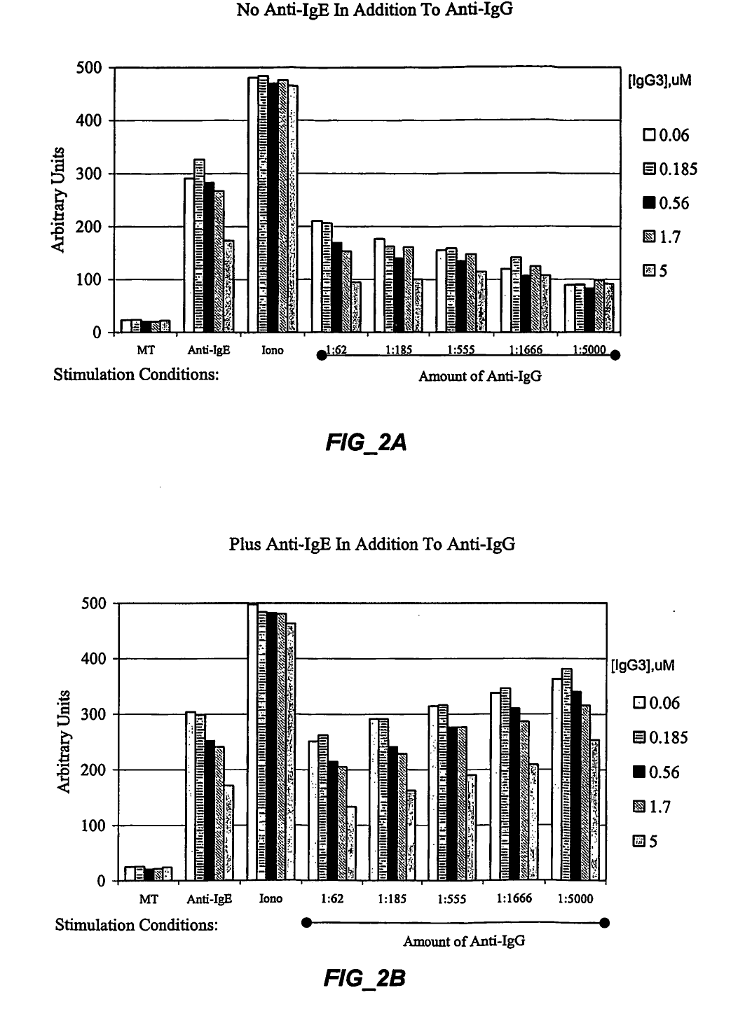

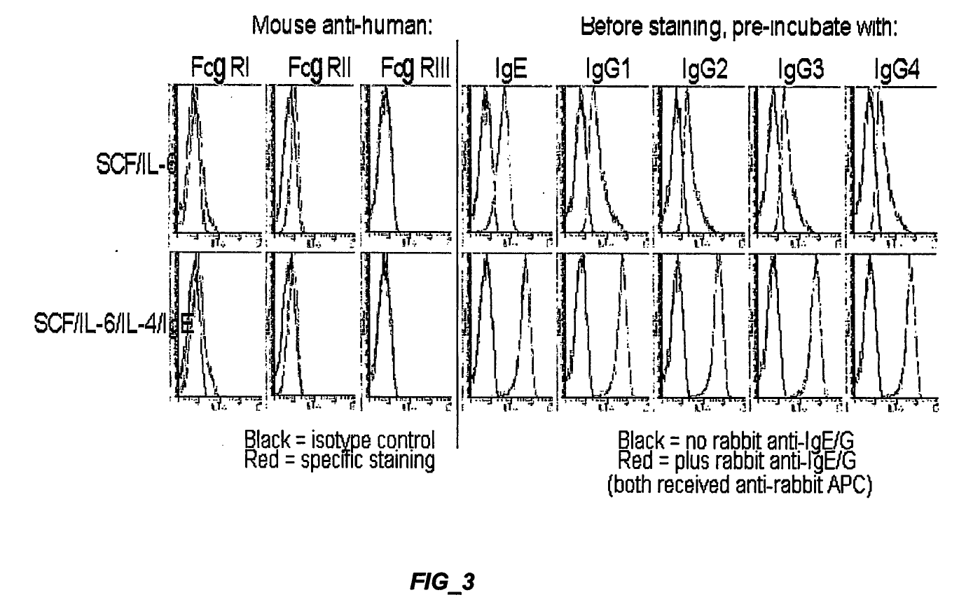

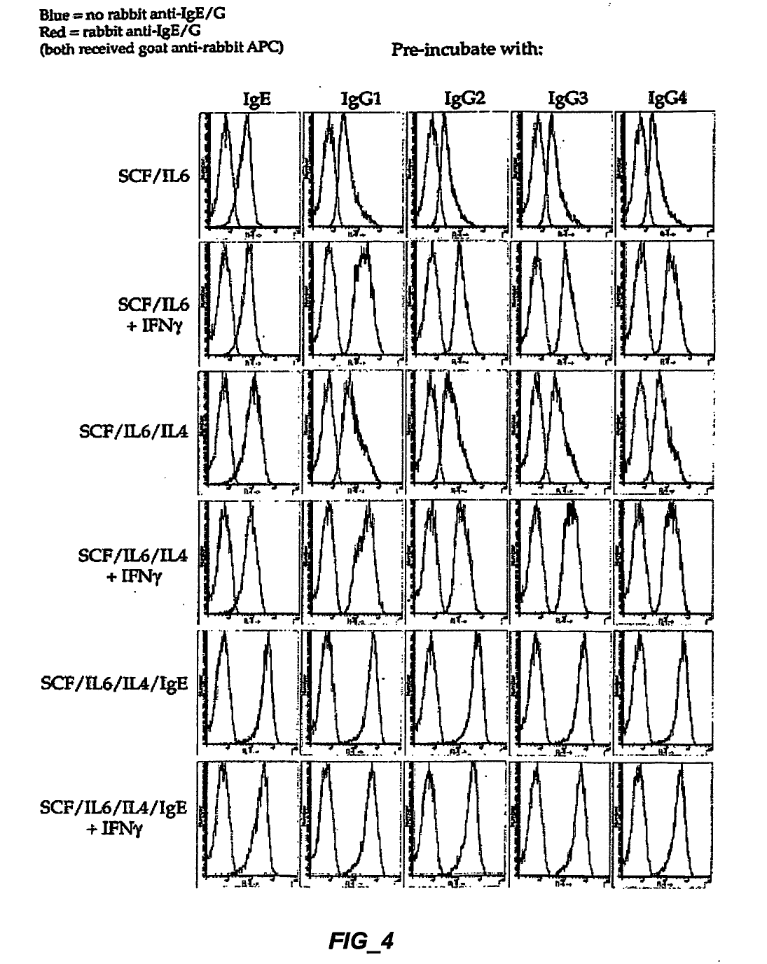

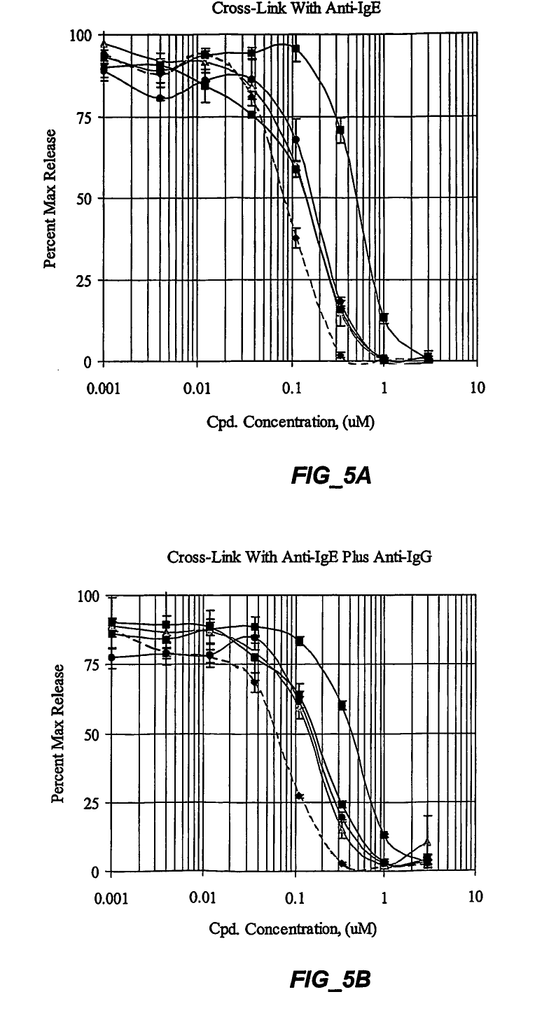

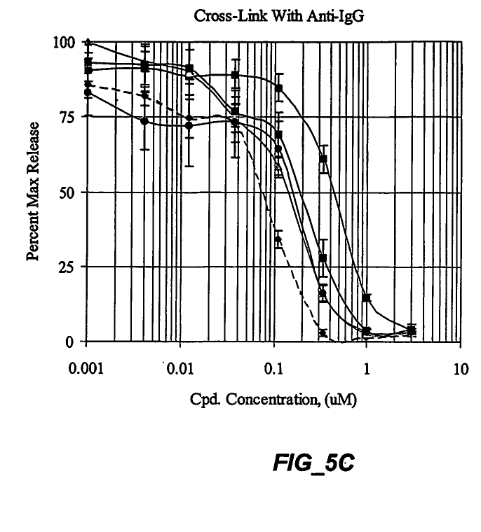

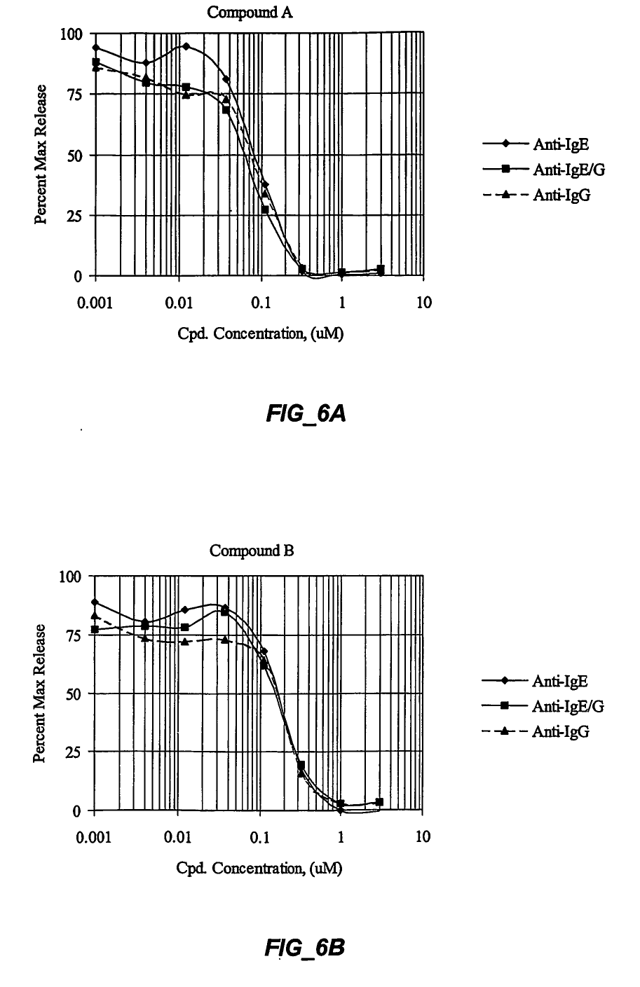

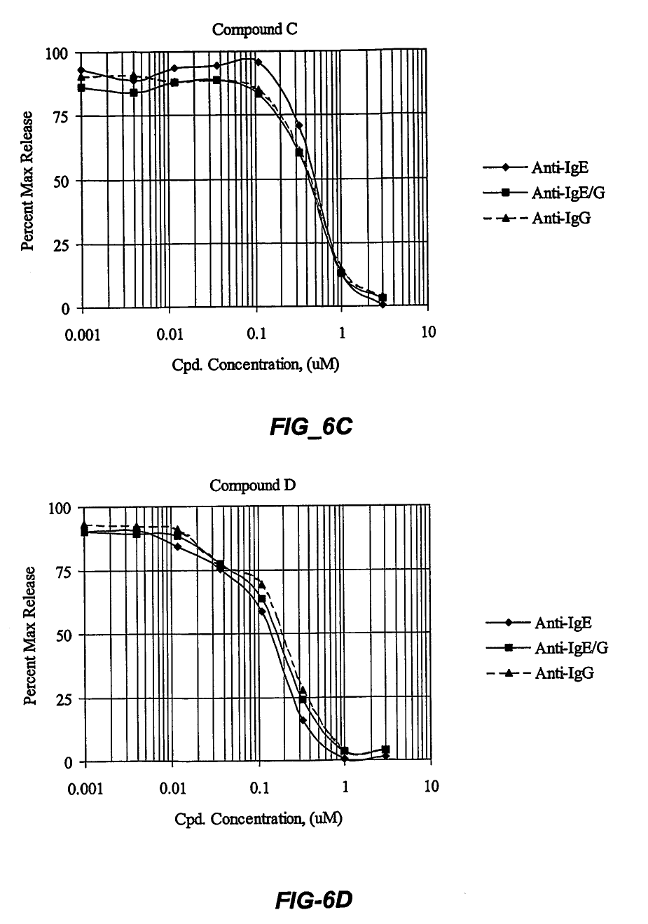

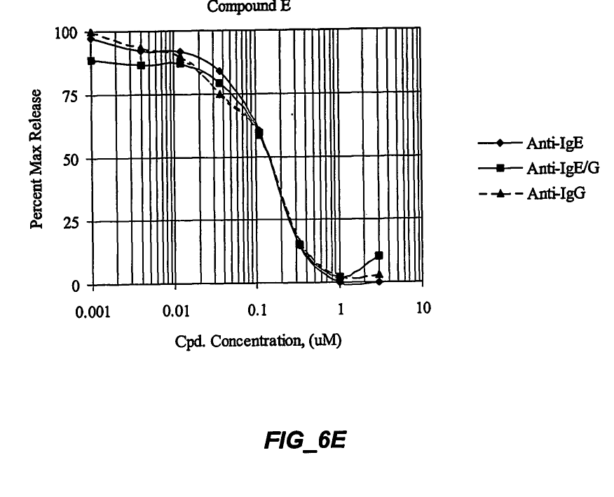

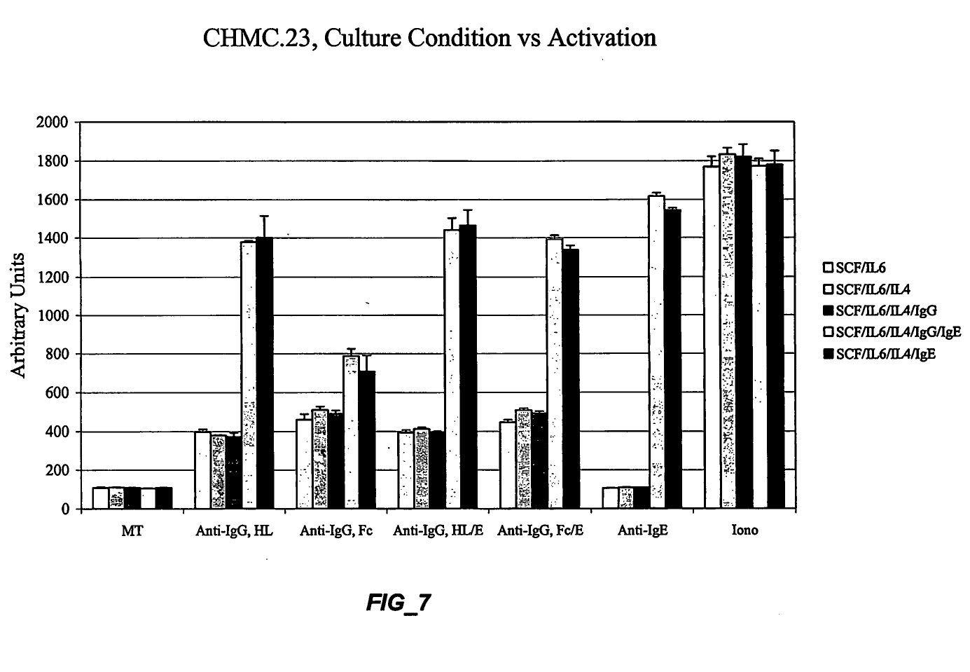

### DETAILED DESCRIPTION OF THE PREFERRED EMBODIMENTS

### Definitions

As used throughout the instant application, the following terms shall have the following meanings:

“Identifying” in the context of screening assays means determining whether a candidate compound unknown to possess a particular property of interest possesses the property of interest, as well as confirming that a compound thought or known to possess a particular property of interest possesses the property of interest.

“Fc Receptor” refers to a member of the family of cell surface molecules that binds the Fc portion (containing the specific constant region) of an immunoglobulin. Each Fc receptor binds immunoglobulins of a specific type. For example, the Fcα receptor (“FcαR”) binds IgA, the FcεR binds IgE, and the FcγR binds IgG.

The FcαR family includes the polymeric Ig receptor involved in epithelial tansport of IgA/IgM, the mycloid specific receptor RcαRI (also called CD89), the Fcα/μR and at least two alternative IgA receptors (for a recent review, see Monteiro and van de Winkel, Annu. Rev. Inmunol, advanced e-publication (2003)). The FcαRI is expressed on neutrophils, eosinophils, moncytes/macrophages, dendritic cells and kupfer cells. The FcαRI inclues one alpha chain and the FcR gamma homodimer that bears an activation motif (ITAM) in the cytoplasmic domain and phosphorylates Syk kinase.

The FcεR family includes two types, designated FcεRI and FcεRII (also known as CD23). FcεRI is a high affinity receptor (binds IgE with an affinity of about 10¹⁰M⁻¹) found on mast, basophil and eosinophil cells that anchors monomeric IgE to the cell surface. The FcεRI possesses one alpha chain, one beta chain and the gamma chain homodimer discussed above. The FcεRII is a low affinity receptor expressed on mononuclear phagocytes, B lymphocytes, eosinophils and platelets. The FcεRII comprises a single polypeptide chain and does not include the gamma chain homodimer.

The FcγR family includes three types, designated FcγRI (also known as CD64), FcγRII (also known as CD32) and FcγRIII (also known as CD16). FcγRI is a high affinity receptor (binds IgG1 with an affinity of 10⁸M⁻¹) found on mast, basophil, mononuclear, neutrophil, eosinophil, deudritic and phagocyte cells that anchors monomeric IgG to the cell surface. The FcγRI includes one alpha chain and the gamma chain dimer shared by FcαRI and FcεRI.

The FcγRII is a low affinity receptor expressed on mast cells, neutrophils, monocytes, eosinophils, platelets, and B lymphocytes. The FcγRII includes one alpha chain, and does not include the gamma chain homodimer discussed above.

The FcγRIII is a low affinity (binds IgG1 with an affinity of 5×10⁵M⁻¹) expressed on NK, eosinophil, macrophage, neutrophil, and mast cells. It comprises one alpha chain and the gamma homodimer shared by FcαRI, FcεRI and FcγRI.

Skilled artisans will recognize that the subunit structure and binding properties of these various Fc receptors, cell types expressing them, are not completely characterized. The above discussion merely reflects the current state-of-the-art regarding these receptors (see, e.g., Immunobiology: The Immune System in Health & Disease, 5th Edition, Janeway et al., Eds, 2001, ISBN 0-8153-3642-x, FIG. 9.30 at pp. 371), and is not intended to be limiting with respect to the myriad receptor signaling cascades.

“Mast cell” refers to specialized, differentiated cells derived from hematopoietic stem cells. Generally, mast cells are characterized by metachromatic staining with basic dyes, for example toluidine blue, which bind acidic proteoglycans present in secretory granules. When activated, mast cells degranulate, releasing preformed mediators and enzymes. These substances include biogenic amine histamine; proteases chymase, tryptase, carboxypeptidase, and cathepsin-G; enzyme hexosaminidase; and proteoglycans. In addition, mast cells express a number of characteristic cell surface molecules, including, by way of example and not limitation, FcεRI and FcγRI.

Mast cells are heterogeneous group of cells, and characterized in the art by differing tissue distribution, morphology, histochemistry, mediator content, and response to activation stimuli (Welle, M., J. Leukoc. Biol. 61:233-245 (1997)). Typically, one major basis of differentiating mast cells is the presence of granule-associated proteases, chymotrypsin-like and trypsin-like proteases (i.e., chymase and tryptase). In humans, mast cells of the “mucosal,” “mucosal type,” “airway,” or “airway type” refers to mast cells that are typical of mast cells present in lung or intestinal mucosa. Mucosal mast cells are characterized by specific binding of high affinity IgE and granule release upon cross-linking of IgE receptors with anti-IgE antibody; by having granules containing histamine; by absence of chymase; and by presence of tryptase. Generally, the mucosal mast cells exhibit a tryptase positive/chymase negative phenotype. In addition, the mucosal mast cells are typically positive for surface expression of high affinity IgE receptors, CD54 and CD117, and negative for or have insignificant surface expression of CD15, CD34, CD25 and CD11b.

Mast cells of the “connective tissue-type” or “cutaneous” or “serosal” refers to mast cells that are typical of mast cells present in connective tissues, for example, skin, lymph nodes and intestinal submucosa. Connective tissue-type mast cells are characterized by high affinity binding of IgE and granule release upon cross-linking of IgE receptors with anti-IgE antibody, by having granules containing relatively high levels of histamine as compared to mucosal mast cells; and by presence of both chymase and tryptase. Generally, the connective tissue-type mast cells of the invention exhibit a tryptase/chymase positive phenotype. In addition, connective tissue-type mast cells are typically positive for surface expression of high affinity IgE receptor, CD54, and CD117, and negative for or have insignificant surface expression of CD15, CD34, CD25 and CD11b.

The classifications of mast cells in the foregoing are not to be applied rigidly and are not intended to limit the present invention. Those skilled in the art will understand the limitations of such categorizations and comprehend the scope of mast cells as used herein.

“Basophil cell” refers to specialized cells that originate from hematopoietic stem cells and are heavily granulated. Basophils synthesize many of the same mediators as mast cells, and express the same high affinity Fcε receptors. Thus, basophils also mediate immediate hypersensitivity reactions to antigen. Additionally, basophils participate in cell-mediated hypersensitivity. Unlike mast cells, basophils mature in the bone marrow and circulate in the blood, from which they are recruited to tissue sites of inflammation.

Basophils are characterized by specific binding of high affinity IgE and granule release upon cross-linking of IgE receptors with anti-IgE antibody, and by having granules containing, for example, histamine and heparin. Basophils are typically positive for surface expression of CD11b, CD13 and CD25, but negative for expression of CD14 and CD117 (see Valent, P. et al., Adv. Immunol. 52:335-339 (1992); Agis, H. et al., Immunology 87:535-43 (1996), hereby incorporated by reference). Thus, in one embodiment, the basophils of the invention are CD11b-positive, CD13-positive, and CD25-positive. In another embodiment, the basophils of the invention are CD14-negative and CD117-negative. The basophils may also be CD54 positive.

“IgE” refers to an antibody with an Fc region corresponding to the heavy chain IgE isotype. IgE may be a naturally occuring antibody, an antibody generated by recombinant methods, or a chimeric antibody with an IgE heavy chain of one animal and a light chain of another animal. Preferably, the IgE has the Fc region corresponding to the IgE present in the animal from which the mast cells are isolated. Thus, for human mast cells, the IgE is preferably a human IgE. The Fc region of IgE antibodies binds to high affinity FcεRI receptors present on mast cells and basophil cells.

“IgG” refers to an antibody with an Fc region corresponding to the heavy chain of the IgG isotype. IgGs are generally classified into four subclasses IgG1, IgG2, IgG3 and IgG4. These subclasses are distinguishable based on numbers of disulfide linkages, molecular weight, abundance in serum, ability to activate complement, antibody dependent cell-mediated cytotoxicity, and binding to various FcγR receptors (Galli, S. J. and Lantz, C. S., Allergy in Fundamental Immunology, 4th Ed., Paul W. E. ed., Lippincott-Raven Publishers, New York (1994)). For example, IgG1 and IgG4 bind to high affinity FcγRI. As with other immunoglobulin isotypes, IgGs may be a naturally occuring antibody, an antibody generated by recombinant methods, or a chimeric antibody with an IgG heavy chain of one animal and a light chain of another animal.

“Primed mast cell” refers to a mast cell displaying enhanced activation compared to an unprimed mast cell. Enhanced or increased activation refers to elevated cellular responses to activating stimuli, such as IgG-mediated activation of Fcγ receptors. In the present invention, the primed mast cells may show degranulation at lower concentrations of IgG, show increases in the amount and duration of release of granule contents, and display changes in other downstream cellular events associated with activation of Fcγ receptor signaling pathway. “Primed basophil cell” as used herein is defined similarly as primed mast cells.

A compound that “modulates Fcγ receptor-mediated signaling” or that “modulates Fcγ signaling cascade” has the ability to change or alter downstream cellular events resulting from activation of the signaling pathway. The change may be monitored at the RNA level, for example by quantifying induced downstream transcription products, or at the protein level, for example by quantifying the amount or activity of induced downstream translation products. Alternatively, the downstream cellular event is a change in cellular physiology, for example degranulation or release of lipid mediators and cytokines.

“Fc Receptor-Mediated Degranulation” or “Fc Receptor-Induced Degranulation” refers to degranulation that proceeds via an Fc receptor signal transduction cascade initiated by crosslinking of an Fc receptor.

“IgE-Induced Degranulation” or “FcεRI-Mediated Degranulation” refers to degranulation that proceeds via the IgE receptor signal transduction cascade initiated by crosslinking of FcεRI-bound IgE. The crosslinking may be induced by an IgE-specific allergen or other multivalent binding agent, such as an anti-IgE antibody. In mast and/or basophil cells, the FcεRI signaling cascade leading to degranulation may be broken into two stages: upstream and downstream. The upstream stage includes all of the processes that occur prior to calcium ion mobilization. The downstream stage includes calcium ion mobilization and all processes downstream thereof Compounds that inhibit FcεRI-mediated degranulation may act at any point along the FcεRI-mediated signal transduction cascade. Compounds that selectively inhibit upstream FcεRI-mediated degranulation act to inhibit that portion of the FcεRI signaling cascade upstream of the point at which calcium ion mobilization is induced. In cell-based assays, compounds that selectively inhibit upstream FcεRI-mediated degranulation inhibit degranulation of cells such as mast or basophil cells that are activated or stimulated with an IgE-specific allergen or binding agent (such as an anti-IgE antibody) but do not appreciably inhibit degranulation of cells that are activated or stimulated with degranulating agents that bypass the FcεRI signaling pathway, such as, for example the calcium ionophores ionomycin and A23187.

“IgG-Induced Degranulation” or “FcγR-mediated Degranulation” refers to degranulation that proceeds via the Fcγ signal transduction cascade, typically initiated by crosslinking of FcγR-bound IgG. The crosslinking may be induced by an IgG-specific allergen or another multivalent binding agent, such as an anti-IgG or fragment antibody. Like the FcεRI signaling cascade, in mast and basophil cells the Fcγ signaling cascade also leads to degranulation which may be broken into the same two stages: upstream and downstream. Similar to FcεRI-mediated degranulation, compounds that selectively inhibit upstream Fcγ-mediated degranulation act upstream of the point at which calcium ion mobilization is induced. In cell-based assays, compounds that selectively inhibit upstream Fcγ receptor-mediated degranulation inhibit degranulation of cells such as mast or basophil cells that are activated or stimulated with an IgG-specific allergen or binding agent (such as an anti-IgG antibody or fragment) but do not appreciably inhibit degranulation of cells that are activated or stimulated with degranulating agents that bypass the Fcγ signaling pathway, such as, for example the calcium ionophores ionomycin and A23187.

“Ionophore-Induced Degranulation” or “Ionophore-Mediated Degranulation” refers to degranulation of a cell, such as a mast or basophil cell, that occurs upon exposure to a calcium ionophore such as, for example, ionomycin or A23187.

“Allergen” refers to antigens capable of binding to IgE or IgG antibodies and mediating activation of mast and/or basophil cells. Allergens comprise various compounds that bind to cognate antibodies and activate mast and/or basophil cells. These include, by way of example and not limitation, pollen grains, environmental particulates (e.g. dust mites), proteins associated with foreign particles or foods, insect venoms, chemicals (e.g., food preservatives, disinfectants, insecticides, etc.), pharmaceutical agents (e.g., antibiotics, steroids, non-steroidal anti-inflammatory drugs, anesthetics, etc.), and the like.

Fc Receptor Signaling Cascades and Priming of Cells with IgE

The present invention relates to methods of identifying compounds capable of modulating Fcγ receptor signaling cascades involved in degranulation of mast and/or basophil cells. Both mast and basophil cells play a central role in allergen-induced disorders, including, for example, allergic rhinitis and asthma. Upon exposure allergens, which may be, among other things, pollen or parasites, allergen-specific antibodies are synthesized by B-cells activated by IL-4 (or IL-13). These allergen-specific IgE antibodies bind to the high affinity FcεRI while IgG antibodies bind Fcγ receptors. Upon binding of antigen, the receptors are cross-linked and the IgE or IgG receptor signal transduction pathway is activated, which leads to degranulation of the cells and consequent release and/or synthesis of a host of chemical mediators, including histamine, proteases (e.g., tryptase and chymase), lipid mediators such as leukotrienes (e.g., LTC4), platelet-activating factor (PAF) and prostaglandins (e.g., PGD2), and a series of cytokines, including TNF-α, IL-4, IL-13, IL-5, IL-6, IL-8, GMCSF, VEGF and TGF-β. The release and/or synthesis of these mediators from mast and/or basophil cells accounts for the early and late stage responses induced by allergens, and is directly linked to downstream events that lead to a sustained inflammatory state.

The molecular events in the FcεRI and FcγR signal transduction pathways that lead to release of preformed mediators via degranulation and release and/or synthesis of other chemical mediators are well-known. Generally, the FcεRI is a heterotetrameric receptor composed of an IgE-binding alpha-subunit, a beta subunit, and two gamma subunits (gamma homodimer). Cross-linking of FcεRI-bound IgE by multivalent binding agents (including, for example IgE-specific allergens or anti-IgE antibodies or fragments) induces the rapid association and activation of the Src-related kinase Lyn. Lyn phosphorylates immunoreceptor tyrosine-based activation motifs (ITAMS) on the intracellular beta and gamma subunits, which leads to the recruitment of additional Lyn to the beta subunit and Syk kinase to the gamma homodimer. These receptor-associated kinases, which are activated by intra- and intermolecular phosphorylation, phosphorylate other components of the pathway, such as the Btk kinase, LAT, and phospholipase C-gamma PLC-gamma. Activated PLC-gamma initiates pathways that lead to protein kinase C activation and Ca²⁺ mobilization, both of which are required for degranulation. FcεRl cross-linking also activates the three major classes of mitogen activated protein (MAP) kinases, i.e. ERK1/2, JNK1/2, and p38. Activation of these pathways is important in the transcriptional regulation of proinflammatory mediators, such as TNF-α and IL-6, as well as the lipid mediator leukotriene CA (LTC4).

The Fcγ receptor signaling cascade, particularly FcγRI, is believed to share some common elements with the FcεRI signaling cascade. Importantly, like FcεRI, the FcγRI includes a gamma homodimer that is phosphorylated and recruits Syk, and like FcεRI, activation of the FcγRI signaling cascade leads to, among other things, degranulation. Other Fc receptors that share the gamma homodimer include, but are not limited to, FcαRI and FcγRIII.

In the present invention, the observation that contacting mast cells with IgE enhances activation mediated by the Fcγ signal transduction pathway, particularly FcγRI, provides a way of generating mast cells useful in screens for identifying compounds that modulate the Fcγ receptor signal transduction pathway, and consequently compounds for treating various disorders related to mast cell activation. In contrast to activation of mast cells by IgE antibody, priming of mast cells with IgE does not appear to require crosslinking of IgE bound to the FcεRI receptor. Thus, IgE-mediated degranulation and mediator release is not a prerequisite for priming mast cells with the IgE antibody. It is to be understood that other cells containing IgE and IgG receptors, such as basophil cells, may be also subjected to the priming step to enhance activation of the Fcγ receptor-mediated signaling pathway.

Accordingly, the present invention provides for methods of priming a mast and/or basophil cell by contacting the cell with IgE antibody and using the cells in methods of identifying compounds that modulate Fcγ receptor-mediated signal transduction pathways. In one aspect, the methods for identifying compounds that modulate Fcγ receptor signaling pathway comprise contacting at least one IgE primed mast and/or basophil cell with a candidate compound in the presence of Fcγ receptor signaling cascade activation, and determining whether the candidate compound modulates the Fcγ receptor-mediated signaling pathway.

In one aspect, by comparing cell activation in the presence and absence of the candidate compound, modulation of Fcγ signaling cascade can be determined. In one embodiment, the modulation is inhibition of Fcγ receptor-mediated cell activation. This effect may be seen as a decrease in cellular responses to Fcγ activation. As provided in detail below, the response of the mast or basophil cell to Fcγ receptor activation is determined by detecting cellular responses downstream of the Fcγ receptor signaling cascade. Downstream cellular events include release of preformed mediators, for example histamine and serotonin; proteoglycans; and proteases such as serine proteases and carboxypeptidase. In a further embodiment, the cellular downstream events are late phase events, which can be evaluated by measuring, by way of example and not limitation, synthesis and release of lipid mediators, and synthesis and release of cytokines.

In a further embodiment, the present invention provides for methods of identifying compounds capable of modulating priming of mast and/or basophil cells by IgE. Generally, the method comprises contacting at least one mast or basophil cell with a candidate compound and IgE antibody. The Fcγ signaling cascade is activated, and the modulation of priming determined by evaluating levels of cell activation.

Increases or decreases of IgE-mediated cell priming is measured. Attenuated or a decreased level of Fcγ receptor-mediated cell activation will indicate an inhibitory effect of the compound on priming. Increased level of Fcγ receptor-mediated cell activation will indicate a stimulatory effect of the compound on priming. By comparing Fcγ receptor-mediated mast or basophil cell activation in presence and absence of candidate compound, modulators of IgE priming can be identified. Contacting or administering the compound before Ig priming will assist in distinguishing between effects of the compound on priming versus cell activation. Furthermore, effect on priming and activation is also distinguishable by examining changes in surface markers, particularly the level of IgG receptors expressed on the cell surface, which in the present invention is found to increase with IgE priming.

Preferably, populations of mast cells are used to screen large numbers of candidate compounds in the methods described herein, particularly when high throughput screens are carried out. The population of mast and/or basophil cells may contain 10⁶-10¹¹ cells. Thus, the populations of cells used for screening may comprise about 10⁶ cells, preferably 10⁹ cells, more preferably 10¹⁰ cells, and most preferably 10¹¹ cells.

Sources of Mast and Basophil Cells for Screening

The mast cells and basophil cells for use in the present invention may be obtained in a variety of ways known to the skilled artisan. These include isolation of cells from tissues, or culturing the cells from progenitor cells by suitable treatment with growth factors and cytokines. Isolation of mast and basophil cells from tissues typically involves enzyme-mediated tissue dispersion and subsequent enrichment by gradient sedimentation, such as single Percoll gradient or multiple Percoll gradients (Patella, V. et al., J Immunol. 154(6):2855-2865 (1995); Stankiewicz, M., et al, Int J Parasitol. 24(2):307-309 (1994)). In another aspect, cell sorting is used to enrich and/or purify the cells. Cellular parameters useful for obtaining enriched population of mast or basophil cells include density, granularity, size, and autofluorescence. Other methods include positive/negative selection by fluorescence activated cell sorting (FACS) using antibodies directed to mast or basophil cell markers, preferably cell surface molecules. Alternatively, negative selection by selective removal of non-mast or non-basophil cells by killing with complement or selective attachment to substrates offer alternative basis for preparing enriched or purified population of cells (Valent, P. and Bettelheim, P., Adv. Immunol. 52:333-423 (1992)).

In another aspect, mast cells and/or basophil cells are generated in vitro from hematopoietic stems cells using media containing combinations of growth factors and cytokines. Typically, umbilical cord blood, bone marrow, or fetal liver serves as sources of hematopoietic stem cells. Various in vitro methods for expanding mast or basophil cells are known to the skilled artisan. For example, mast cells may be generated in a single step by contacting CD34+ positive stem cells with various combinations of stem cell factor (SCF), flt-3 ligand, and cytokines that expand populations of progenitor cells and also induce differentiation into various mast cell types. For example, SCF and IL-6 induces stem cells to expand and differentiate into a population of mast cells displaying predominantly tryptase positive cells and a minor percentage of chymase positive cells (Saito, H. et al., Int. Arch. Allergy Immunol. 107:63-65 (1995)). Additional extracellular factors used to expand cultured mast cells include prostaglandin and thrombopoietin (Saito, H. et al., J Immunol. 157(1):343-350 (1996); Gilmore, G. L. et al., Exp Hematol. 28(11):1297-1305 (2000)).

Preferably, the cultured mast or basophil cells are expanded from a population of progenitor cells, as described in copending U.S. application Ser. No. 10/053,355, filed Nov. 8, 2001, the entire contents of which is incorporated herein by reference. Generally, a population of CD34+ positive cells is contacted with SCF and flt-3 ligand to generate a population of progenitor cells capable of differentiating into mast or basophil cells. Subsequently, the expanded population of progenitor cells is contacted with SCF and a cytokine suitable for inducing differentiation of the expanded progenitor cells into mast or basophil cells. Presence of cytokine IL-6 induces differentiation into mucosal mast cells; presence of IL-4 induces differentiation into connective tissue-type mast cells; and presence of IL-3 induces differentiation into basophil cells.

In another aspect, the type of mast cells used in the assay will have a defined phenotype. Thus, in one embodiment, the mast cells in the screens may comprise mucosal mast cells, which for human mast cells typically have a tryptase positive and chymase negative phenotype. In another embodiment, the mast cells in the screens may comprise connective tissue-type mast cells, which for human mast cells typically have tryptase and chymase positive phenotype. The known heterogeneity of mast cells with regard to granule content and tissue distribution suggests that mast cells differ with respect to their role in different allergic and inflammatory responses. Thus, mucosal type mast cells typical of the lung and intestinal submucosa, and connective tissue-type mast cells typical of the skin, intestinal submucosa, and lymph nodes, may provide mast cell types that respond differently to candidate compounds. This allows identification of compounds selective for particular types of allergic and inflammatory responses.

The origin of the mast or basophil cells used in the assay will depend, in part, on the desired use for the compounds and will be apparent to those of skill in the art. For example, if the compounds will be used to treat or prevent a particular disease in humans, a convenient source of mast or basophil cells is a human or other animal that constitutes an accepted or known clinical model for the particular disease. Thus, depending upon the particular application, the mast or basophil cells may be derived from a wide variety of animal sources, ranging from, for example, lower mammals such as mice and rats, to dogs, sheep and other mammals commonly employed in clinical testing, to higher mammals such as monkeys, chimpanzees and apes, to humans. Specific examples of cells suitable for carrying out the in vitro assays include, but are not limited to, rodent or human mast cells, primary mouse mast cells (such as bone marrow-derived mouse mast cells “BMMC”) and primary human mast cells isolated from cord blood (“CHMC”) or other tissues such as lung (see, e.g., Demo, S. D. et al., Cytometry 36(4):340-348 (1999)).

In accordance with the above, the mast or basophil cells may be isolated from animals having variations or defects in processes related to mast or basophil cell activation. These may be naturally occurring genetic defects or allelic variants in an animal population, or animals genetically engineered to have targeted disruptions or misexpression of genes involved in mast or basophil cell activation (see, e.g., Matsuda, H et al., Int Immunol. 9(3):461-466 (1997); Li, A. et al., Clin Sci (Lond). 93(3):279-286 (1997); and Dombrowicz, D. et al., Immunity. 8(4):517-529 (1998)). For example, animals carrying disruptions or mutations in Kit receptor may be used (Galli, S. J. et al., Ann. N.Y. Acad. Sci. 664:69-98 (1992)).

Priming and Activation of Mast or Basophil Cells

Mast or basophil cells, isolated from tissues or produced in culture, are contacted with IgE to prime the cells, which renders the cell susceptible to Fcγ receptor-mediated activation. A variety of IgE antibodies are useful for this purpose. The IgE antibodies may be polyclonal or monoclonal, or comprise the Fc portion of IgE prepared recombinantly or by proteolysis of IgE molecules. The IgE may be preparations of naturally occurring IgE; IgE made recombinantly; chimeric antibodies containing Fc region of one animal species and light chain of another animal species; or an antibody of one species made in another animal species, such as completely “humanized” antibodies made in mouse. Generally, chimeric antibodies are produced by cells engineered with variable heavy chain (VH) and variable light chain (VL) gene segments of the desired specificity from one animal species and spliced to constant heavy chain (CH) and constant light chain (CL) gene segments of another animal species, such as human (Morrison, S. L. et al., Proc. Natl Acad. Sci. USA 81:6851-6866 (1984); Neuberger, M. S. et al., Nature 312:604-608 (1984); Takeda, S. et al., Nature 314:452-454 (1985); Boss, U.S. Pat. No. 4,816,397; Cabilly, U.S. Pat. No. 4,816,567). In some cases, whole antibodies, monoclonal or polyclonal, of one animal may be made by tranferring the complement of its antibody genes (e.g., VH, VL, CH and CL) into cells of another animal species in which the endogenous antibody genes have been inactivated or deleted. Various methods are known in the art for generating chimeric antibodies, including, but not limited to, phage display libraries (Vaughan, T. J., Nature Biotechnol. 16(6):535-539 (1998)), and cloning and expressing antibody gene segments in mammalian or plant cells (Shearman, C. W. et al., J. Immunol. 146(3):928-935 (1991); Norderhaug, L. et al., J. Immunol Methods. 204(1):77-87 (1997); Luiten, R. M. et al., Hum Antibodies 8(4):169-180. (1997)). Transgenic animals whose antibody genes have been replaced by antibody genes of another animal species are described in Tomizuka, K. et al., Proc. Natl. Acad. Sci. USA 97(2):722-727 (2000) and Lonberg, N. et al., Nature 368:856-859 (1994)).

Generally, although IgE antibodies of one species may be used to prime mast or basophil cells of another animal species, preferably the IgE antibodies used for priming have the Fc region derived from the same animal species from which the cells are obtained. For example, human mast or basophil cells are primed with human IgE antibodies or with chimeric antibodies containing a human Fc region, while mouse mast or basophil cells are primed with mouse IgE antibodies or with chimeric antibodies containing a mouse Fc region. The cells are contacted with IgE at a sufficient concentration and incubation time to enhance Fcγ receptor-mediated cell activation. Generally, an IgE concentration of up to about 5 ug/ml may be used, with incubation periods ranging from about 3 to about 7 days. By way of example and not limitation, priming of cultured human mast cells is accomplished in culture with about 200 ng/ml IgE for about 3 days. Mast cells treated under such conditions remain primed for about 3 to about 8 days. By contacting mast or basophil cells with different IgE concentrations and incubation times and subsequent evaluation of Fcγ receptor-mediated cell activation, the skilled artisan can determine the range of effective conditions required for priming.

Once primed, the cells are activated by various compounds capable of activating Fcγ receptor-mediated signaling pathway. Preferably, to limit complications from activation of FcεRI receptor-mediated signaling, Fcγ receptors may be selectively activated. In one aspect, activation is through use of agents that crosslink Fcγ receptors. Typically, receptor crosslinking and corresponding activation of the signaling cascade is achieved by combination of an IgG antibody and a cognate anti-IgG antibody capable of crosslinking the receptor bound IgG molecule. These anti-IgG antibodies include, by way of example and not limitation, an anti-IgG antibody directed against the heavy and light chain of the IgG antibody. Alternatively, the IgG may comprise a chimeric protein containing an Fc region linked to a heterologous protein in which the crosslinking antibody reacts with the heterologous portion.

Another method of activating mast cells comprises contacting the cells with an antibody raised directly against the extracellular region of Fcγ receptor and treating the cells with antibodies directed against the anti-Fcγ receptor antibody. The anti-anti Fcγ antibody causes additional crosslinking interactions which facilitate activation of the signaling pathway. In a variation of this technique, contacting the cells with F(ab)₂ fragments obtained from anti-Fcγ receptor antibodies and reacting with an antibody directed against the F(ab)₂ fragment may also produce effective receptor crosslinking and subsequent activation of signal transduction. Though use of anti-receptor antibodies or F(ab)₂ fragments raised against a specific type of Fcγ receptor (e.g., FcγRI, FcγRII, or FcγRII), specific Fcγ signaling pathways may be activated and examined.

In another aspect, activation of Fcγ receptor signaling pathway is achieved with IgG antibodies directed against a hapten, for example dinitrophenol (DNP). Mast cells contacted with anti-hapten IgG are treated with a protein carrier conjugated to a plurality of the specified hapten (e.g., DNP-bovine serum albumin), thus resulting in receptor crosslinking and subsequent activation of the Fcγ receptor signaling cascade. In place of protein carriers, other substrates with attached haptens may be used, including, by way of example and not limitation, dextran or latex beads. In a further aspect, activation is induced by use of an IgG antibody reactive with a specific allergen. Various allergens known to react with IgG antibodies include, by way of example and limitation, lactalbumin and lactaglobulin in cow's milk (Jarvinen, K. M., Int. Arch. Allergy Immunol. 126(2):111-118 (2001)); birch pollen (Denepoux, S. et al., FEBS Lett. 465(1):39-46 (2000); grass pollen (Michils, A. et al., Clin Exp Allergy. 29(6):832-839 (1999); and egg white ovomucoid (Zhang, J. W. et al., Biochem. Biophys. Res. Commun. 253(1):124-127 (1998). As the foregoing illustrates, suitable Fcγ receptor activating agents will be apparent to those of skill in the art.

Assays for Determining Mast or Basophil Cell Activation and Fcγ Receptor Signaling

The primed mast or basophil cells may be subjected to any screening technique known in the art for determining whether compounds modulate Fcγ receptor signaling. Suitable assays for modulation of Fcγ receptor-mediated degranulation are similar to those used for detecting degranulation via the Fcε receptor pathway. In one typical assay, the amount of a chemical mediator or other chemical agent released and/or synthesized as a consequence of activating the Fcγ signaling cascade may be quantified using standard techniques and compared to the amount of the mediator or agent released from control cells (i.e., cells that are stimulated but that are not exposed to test compound).

As will be recognized by skilled artisans, the mediator or agent quantified is not critical. The only requirement is that it be a mediator or agent released and/or synthesized as a consequence of initiating or activating the Fc receptor signaling cascade. For example, activation of the Fcγ signaling cascade in mast and/or basophil cells leads to numerous downstream events. For example, activation of the Fcγ signal cascade leads to the immediate release (i.e., within 1-3 min. following receptor activation) of a variety of preformed chemical mediators and agents via degranulation. Thus, in one embodiment, the mediator or agent quantified may be specific to granules (i.e., present in granules but not in the cell cytoplasm generally). Examples of granule-specific mediators or agents that can be quantified to determine and/or confirm the activity of a candidate compound include, but are not limited to, granule-specific enzymes such as hexosaminidase, tryptase, and chymase, and granule-specific components such as histamine and serotonin. Assays for quantifying such factors are well-known, and in many instances are commercially available. For example, tryptase and/or hexosaminidase release may be quantified by incubating the cells with cleavable substrates that fluoresce upon cleavage and quantifying the amount of fluorescence produced using conventional techniques. Such cleavable fluorogenic substrates are commercially available. For example, the fluorogenic substrates Z-Gly-Pro-Arg-AMC (Z=benzyloxycarbonyl; AMC=7-amino-4-methylcoumarin; BIOMOL Research Laboratories, Inc., Plymouth Meeting, Pa. 19462, Catalog No. P-142) and Z-Ala-Lys-Arg-AMC (Enzyme Systems Products, a division of ICN Biomedicals, Inc., Livermore, Calif. 94550, Catalog No. AMC-246) can be used to quantify the amount of tryptase released. The fluorogenic substrate 4-methylumbelliferyl-N-acetyl-β-D-glucosaminide (Sigma, St. Louis, Mo., Catalog No. 69585) can be used to quantify the amount of hexosaminidase released. Histamine release may be quantified using a commercially available enzyme-linked immunosorbent assay (ELISA) such as Immunotech histamine ELISA assay #IM2015 (Beckman-Coulter, Inc.). Any of these assays may be used to determine or confirm the activity of candidate compounds.

Degranulation is only one of several responses initiated by the Fcγ signaling cascade. In addition, activation of the signaling pathway leads to the de novo synthesis and release of cytokines and chemokines such as IL-4, IL-5, IL-6, TNF-α, IL-13 and MIP1-α, and release of lipid mediators such as leukotrienes (e.g., LTC4), platelet activating factor (PAF) and prostaglandins. Accordingly, the candidate compounds may also be assessed for activity by quantifying the amount of one or more of these mediators released and/or synthesized by activated cells.

Unlike the granule-specific components discussed above, these “late stage” mediators are not released immediately following activation of the Fcγ signaling cascade. Accordingly, when quantifying these late stage mediators, care should be taken to insure that the activated cell culture is incubated for a time sufficient to result in the synthesis (if necessary) and release of the mediator being quantified. Generally, PAF and lipid mediators such as leukotriene C4 are released 3-30 min. following Fcγ activation. The cytokines and other late stage mediators are released approximately 4-8 hrs after receptor activation. Incubation times suitable for a specific mediator will be apparent to those of skill in the art. Specific guidance and assays are provided in the Examples section.

The amount of a particular late stage mediator released may be quantified using any standard technique. In one embodiment, the amount(s) may be quantified using ELISA assays. ELISA assay kits suitable for quantifying the amount of TNFα, IL4, IL-5, IL-6 and/or IL-13 released are available from, for example, Biosource International, Inc., Camarillo, Calif. 93012 (see, e.g., Catalog Nos. KHC3011, KHC0042, KHC0052, KHC0061 and KHC0132). ELISA assay kits suitable for quantifying the amount of leukotriene C4 (LTC4) released from cells are available from Cayman Chemical Co., Ann Arbor, Mich. 48108 (see, e.g., Catalog No. 520211).

In addition to the FcεRI or FcγR degranulation pathways discussed above, degranulation of mast and/or basophil cells can be induced by other agents. For example, ionomycin, a calcium ionophore that bypasses the early FcεRI or FcγR signal transduction machinery of the cell, directly induces a calcium flux that triggers degranulation. Activated PLCγ initiates pathways that lead to, among other things, calcium ion mobilization and subsequent degranulation. This Ca²⁺ mobilization is triggered late in the Fc receptor signal transduction pathway. As mentioned above, ionomycin directly induces Ca²⁺ mobilization and a Ca²⁺ flux that leads to degranulation. Other ionophores that induce degranulation in this manner include A23187. The ability of granulation-inducing ionophores such as ionomycin to bypass the early stages of the Fcγ receptor signaling cascades may be used as a counter screen to identify active compounds of the invention that specifically exert their degranulation-inhibitory activity by blocking or inhibiting the early Fcγ receptor signaling cascades, as discussed above. Compounds which specifically inhibit such early Fcγ receptor-mediated degranulation inhibit not only degranulation and subsequent rapid release of histamine, tryptase and other granule contents, but also inhibit the pro-inflammatory activation pathways causing the release of TNFα, IL-4, IL-13 and the lipid mediators such as LTC4. Thus, compounds which specifically inhibit such early Fcγ receptor-mediated degranulation block or inhibit not only acute atopic or Type I hypersensitivity reactions, but also late responses involving multiple inflammatory mediators.

Other suitable methods for determining Fcγ receptor-mediated mast cell activation include examining the biochemical signaling reactions that occur upon Fcγ signal transduction. This includes determining the phosphorylation states of kinases and kinase substrates involved in Fcγ receptor signal transduction. In one aspect, the phorphorylation of the ITAMs present on Fcγ receptor gamma chains may be examined. These motifs are phosphorylated by tyrosine kinase Lyn and may be detected by antibodies directed to phosphorylated ITAM sequences, or immuneprecipitation of Fcγ receptor in cells exposed to radioactive substrate ATP. It is also known that tyrosine kinases Lyn and Syk are autocatalytic and are phosphorylated upon activation. Thus, phosphorylation states of these kinases may be similarly determined.

In yet another embodiment, the assays for mast cell activation may use lipophilic styryl dyes such as RH414, RH795, FM1-43, and FM 4-64 (see U.S. application Ser. No. 10/053,355, incorporated herein by reference). These lipophilic dyes reversibly bind the outer leaflet of cell membranes and become trapped in endocytic vesicles following vesicle recycling. Upon degranulation, the dyes are released into the medium resulting in loss of dye from the cells. Because the dyes fluoresce more intensely in the lipid environment of the endocytic vesicle, the presence of dye in the cells provides a measure of degranulation (Betz, H. et al., Curr. Opin. Neurobiol. 6:365-371 (1996); Haugland, P. T., Handbook of Fluorescent Probes and Research Products, 6th Ed, (1996) (see Chapter 17), incorporated herein by reference.

Another dye-based assay useful for measuring degranulation following cell activation are flurophores linked to weak base. These dyes are cell membrane permeant, and it is theorized that the dyes are protonated in the acidic environment of exocytic granules, which results in retention of the dye in the vesicle. Cell activation results in release of the dye from the cell. Thus, changes in dye release will reflect changes in cell activation. Suitable dyes include LYSOTRACKER® red, LYSOTRACKER® green, and LYSOTRACKER® blue. (Haugland, P. T., supra, Chapter 17; Hafler T. et al., Cell Calcium 19(2):157-165 (1996)).

In yet a further embodiment, the assay for determining mast or basophil cell activation uses the ability of Annexin V to bind exocytic granules exposed to the external environment (Demo, S. D., Cytometry. 36(4):340-348 (1999), incorporated herein by reference). Following degranulation, mast cells show an increase in Annexin binding proportional to the time and intensity of degranulation. Consequently, changes in Annexin binding provide a measure of mast or basophil cell activation. Annexin is commercially available and may be directly labeled with a fluorophore, such as fluorescein isothiocyanate (FITC), TRITC, CY5, or other fluorescent compounds known in the art. Alternatively, the Annexin contains a first label, such as a ligand (e.g., biotin), and a cognate binding partner (e.g., streptavidin) labeled with a detectable molecule (e.g., fluorophore, enzyme, etc.) is used to detect presence of the first label. It is understood that these and other assays for measuring cell activation are well known to the skilled artisan.

Candidate Compounds and Screening of Compounds

The compounds screened can range from small organic molecules to large polymers and biopolymers, and can include, by way of example and not limitation, small organic compounds, saccharides, carbohydrates, polysaccharides, lectins, peptides and analogs thereof, polypeptides, proteins, antibodies, oligonucleotides, polynucleotides, nucleic acids, etc. In one embodiment, the candidate compounds screened are small organic molecules having a molecular weight in the range of about 100-2500 daltons. Such candidate molecules will often comprise cyclical structures composed of carbon atoms or mixtures of carbon atoms and one or more heteroatoms and/or aromatic, polyaromatic, heteroaromatic and/or polyaromatic structures. The candidate agents may include a wide variety of functional group substituents. In one embodiment, the substituent(s) are independently selected from the group of substituents known to interact with proteins, such as, for example, amine, carbonyl, hydroxyl and carboxyl groups.

The candidate compounds may be screened on a compound-by-compound basis or, alternatively, using one of the myriad library techniques commonly employed in the art. For example, synthetic combinatorial compound libraries, natural products libraries and/or peptide libraries may be screened using the assays of the invention to identify compounds that modulate Fcγ receptor-mediated mast or basophil cell activation. Although candidate compounds may be screened on a compound-by-compound basis, it may be more convenient to screen large numbers of candidate compounds simultaneously using one of the many library screening methodologies known in the art. One art-known approach uses recombinant bacteriophage to produce large libraries of peptides that can then be screened in a variety of formats. Using such phage methods, very large libraries of candidate peptides can be constructed (e.g., 10⁶-10⁸ peptides). Methods for constructing and screening such “phage display” libraries are described, for example, in Scott and Smith, Science 249:386-390 (1990); Cwirla, S. E. et al., Proc. Natl. Acad. Sci. USA 87:6378-6382 (1990); Devlin, J. J. et al., Science 249:404-406 (1990); U.S. Pat. No. 5,427,908; U.S. Pat. No. 5,432,018; U.S. Pat. No. 5,580,717 and U.S. Pat. No. 5,723,286, the disclosures of which are incorporated herein by reference. Other non-limiting examples of recombinant library methodologies that may be used in connection with the assays of the invention are described in U.S. Pat. No. 6,156,571; U.S. Pat. No. 6,107,059 and U.S. Pat. No. 5,733,731, the disclosures of which are incorporated herein by reference.

A second art-known approach uses chemical methods to synthesize libraries of compounds, such as small organic compounds, peptides and/or peptide analogs, attached to beads or wafers that can then be conveniently screened. The libraries may be encoded or non-encoded. Methods of synthesizing such immobilized libraries, as well as methods of screening the libraries are described, for example, in Houghten, Proc. Natl. Acad. Sci. USA 82:5131-5735 (1985); Geysen et al., Molecular Immunology 23:709-715 (1986); Geysen et al., J. Immunologic Method 102:259-274 (1987); Frank and Döring, Tetrahedron 44:6031-6040 (1988); Fodor et al., Science 251:767-773 (1991); Furka et al., 4th International Congress of Biochemistry, Volume 5, Abstract FR:013 (1988); Furka, Int. J. Peptide Protein Res. 37:487-493 (1991); Frank, Tetrahedron 48:9217-9232 (1991); Needels et al., Proc. Natl. Acad. Sci. USA 90:10700-10704 (1993); DeWitt et al., Proc. Natl. Acad. Sci. USA 90:6909-6913 (1993); Frank et al., Biorg. Med. Chem. Lett. 3:425-430 (1993); Ohlmeyer et al., Proc. Natl. Acad. Sci. USA 90:10922-10926 (1993); WO 92/00252; WO 9428028; U.S. Pat. No. 6,329,143; U.S. Pat. No. 6,291,183; U.S. Pat. No. 5,885,837; U.S. Pat. No. 5,424,186; U.S. Pat. No. 5,384,261; U.S. Pat. No. 6,165,717; U.S. Pat. No. 6,143,497; U.S. Pat. No. 6,140,493; U.S. Pat. No. 5,789,162; U.S. Pat. No. 5,770,358; U.S. Pat. No. 5,708,153; U.S. Pat. No. 5,639,603; U.S. Pat. No. 5,541,061; U.S. Pat. No. 5,525,735; U.S. Pat. No. 5,525,734; U.S. Pat. No. 6,261,776; U.S. Pat. No. 6,239,273; U.S. Pat. No. 5,846,839; U.S. Pat. No. 5,770,455; U.S. Pat. No. 5,770,157; U.S. Pat. No. 5,609,826; U.S. Pat. No. 6,001,579; U.S. Pat. No. 5,968,736; U.S. Pat. No. 5,962,337; U.S. Pat. No. 5,789,172; U.S. Pat. No. 5,721,099; U.S. Pat. No. 5,565,324; U.S. Pat. No. 5,010,175; and U.S. Pat. No. 4,631,211, the disclosures of which are incorporated herein by reference. For reviews of some of these techniques, see Ellman et al., Account, Chem. Res. 29:132-143 (1996); Gallop et al., J. Med. Chem. 37:1233-1251 (1994); Gordon et al., J. Med. Chem. 37:1385-1401 (1994). Non-limiting examples of solid-phase chemical synthesis strategies and conditions useful for synthesizing combinatorial libraries of small organic and other compounds may be found in Bunin, The Combinatorial Index, Academic Press, London, England (1998) (see, e.g., Chapters 1-5) and Hemikens et al., Tetrahedron 52:4527-4554 (1996), as well as the references cited therein, the disclosures of which are incorporated herein by reference.

Another art-known approach utilizes solution-phase chemical synthesis techniques to synthesize libraries of compounds, such as, for example, libraries of small organic compounds, which may then be screened in the assays of the invention. Methods for synthesizing and screening such solution-phase libraries are well-known and are described, for example, in Bunin, The Combinatorial Index, Academic Press, England (1998) (see, e.g., Chapter 6); WO 95/02566; U.S. Pat. No. 5,962,736; U.S. Pat. No. 5,766,481; U.S. Pat. No. 5,736,412 and U.S. Pat. No. 5,712,171, and the references cited therein; the disclosures of which are incorporated herein by reference. Additional review articles, references, patents and books describing myriad techniques for synthesizing and screening libraries of compounds can be found at Lebl and Leblova: Dynamic Database of References in Molecular Diversity, Internet www.5z.com (see especially the diversity information pages at www.5z.com/divinfo).

The mast or basophil cell may be contacted with the candidate compounds before, during, or after activating the Fcγ receptor signaling pathway. Preferably, in methods of identifying compounds that modulate Fcγ receptor signaling cascade, the cells are contacted with the candidate compounds prior to activation of the signaling pathway and/or in presence of Fcγ receptor activation. In some instances, it is desirable to determine the effect of the candidate compounds on an unactivated, primed or unprimed cell, or where activation is inhibited. In methods of identifying compounds that modulate IgE-mediated priming of cells, the mast or basophil cells are preferably contacted with candidate compounds prior to priming the cells with IgE, to distinguish effects of the compound on priming versus cell activation.

The candidate agent can be added exogenously to the cells or can be administered into the cells. For compounds that readily traverse cell membranes, the compound may be administered to the cell by contacting the cell with the compound. In one embodiment, such compounds may be administered using well-known retroviral vectors and infection techniques pioneered by Richard Mulligan and David Baltimore with Psi-2 lines and analogous retroviral packaging systems based upon NIH 3T3 cells (see Mann et al., Cell 33:153-159 (1993), the disclosure of which is incorporated herein by reference). Such helper-defective packaging cell lines are capable of producing all of the necessary trans proteins (gag, pol and env) required for packaging, processing, reverse transcribing and integrating genomes. Those RNA molecules that have in cis the ψ packaging signal are packaged into maturing retrovirions. Virtually any of the art-known retroviral vectors and/or transfection systems may be used. Specific non-limiting examples of suitable transfection systems include those described in WO 97/27213; WO 97/27212; Choate et al., Human Gene Therapy 7:2247-2253 (1996); Kinsella et al., Human Gene Therapy 7:1405-1413 (1996); Hofmann et al., Proc. Natl. Acad. Sci. USA 93:5185-5190 (1996); Kitamura et al., Proc. Natl. Acad. Sci. USA 92:9146-9150 (1995); WO 94/19478; Pear et al., Proc. Natl. Acad. Sci. USA 90:8392-8396 (1993); Mann et al., Cell 33:153-159 (1993) and the references cited in all of the above, the disclosures of which are incorporated herein by reference. Specific non-limiting examples of suitable retroviral vector systems include vectors based upon murine stem cell virus (MSCV) as described in Hawley et al., Gene Therapy 1:136-138 (1994); vectors based upon a modified MFG virus as described in Rivere et al., Genetics 92:6733 (1995); pBABE as described in WO 97/27213 and WO 97/27212; and the vectors depicted in FIG. 11 of WO 01/34806, the disclosures of which are incorporated herein by reference. Other suitable vectors and transfection techniques, or carrier systems for administration into cells, including methods such as calcium phosphate, DEAE dextran, liposomes, electroporation, biolistic particle bombardment, micro- and nanoparticles, and the like, are well known in the art.

Ideally, the ability to inhibit the release of all desired types of mediators will reside in a single compound. However, mixtures of compounds can also be identified that achieve the same result. For example, a first compound which inhibits release of granule specific mediators may be used in combination with a second compound which inhibits the release and/or synthesis of cytokine mediators.

In another embodiment, the method is used to identify candidate compounds that may modulate, in particular to inhibit, Fcγ receptor-mediated mast or basophil cell activation as a therapeutic approach towards the treatment or prevention of diseases characterized by, caused by and/or associated with the release or synthesis of chemical mediators of Fcγ receptor signaling cascades or degranulation. Diseases that are characterized by, caused by or associated with such mediator release, synthesis or degranulation, and that can therefore be treated or prevented with the candidate compounds include, by way of example and not limitation, atopy or anaphylactic hypersensitivity or allergic reactions, allergies (e.g., allergic conjunctivitis, allergic rhinitis, atopic asthma, atopic dermatitis and food allergies), low grade scarring (e.g., of scleroderma, increased fibrosis, keloids, post-surgical scars, pulmonary fibrosis, vascular spasms, migraine, reperfusion injury and post myocardial infarction), diseases associated with tissue destruction (e.g., of COPD, cardiobronchitis and post myocardial infarction), diseases associated with tissue inflammation (e.g., irritable bowel syndrome, spastic colon and inflammatory bowel disease), inflammation and scarring.

Kits

The invention also provides kits for carying out the various screening assays and methods of the invention. These kits will typically include IgE antibodies for priming cells; the components required for activating the mast or basophil cells, for example F(ab)₂ directed against FcγRI receptor (particularly human FcγRI) and anti-F(ab)₂ antibodies for crosslinking and receptor activation; and populations of mast and/or basophil cells. The kit may further include components for detecting cell activation, including, substrates for granule-associated proteases (e.g., tryptase or chymase) or enzymes (e.g., hexosaminidase), or ELISA assays for late stage mediators (e.g., leukotrienes and cytokines). Additional components of the kits, include as non-limiting examples, labels, cell culture media, buffers, etc.

The invention having been described, the following examples are offered by way of illustration and not limitation. They are not intended to be exhaustive or to limit the invention to the precise forms disclosed, and obviously many modifications and variations are possible in light of the above teaching.

All patents, patent applications, publications, and references cited herein are expressly incorporated by reference to the same extent as if each individual publication or patent application was specifically and individually indicated to be incorporated by reference.

### EXAMPLES

### Example 1

### Culturing of Human Mast and Basophil Cells

Human mast and basophil cells were cultured from progenitor cells as described below (see also the methods described in copending U.S. application Ser. No. 10/053,355, filed Nov. 8, 2001, the disclosure of which is incorporated herein by reference).

Preparation of STEMPRO-34 Complete Medium

To prepare STEMPRO-34 complete medium (“CM”), 250 mL STEMPRO-34TM serum free medium (“SFM”; GibcoBRL, Catalog No. 10640) was added to a filter flask. To this was added 13 mL STEMPRO-34 Nutrient Supplement (“NS”; GibcoBRL, Catalog No. 10641) (prepared as described in more detail, below). The NS container was rinsed with approximately 10 mL SFM and the rinse added to the filter flask. Following addition of 5 mL L-glutamine (200 mM; Mediatech, Catalog No. MT 25-005-CI and 5 mL 100× penicillin/streptomycin (“pen-strep”; HyClone, Catalog No. SV30010), the volume was brought to 500 mL with SFM and the solution was filtered.

The most variable aspect of preparing the CM is the method by which the NS is thawed and mixed prior to addition to the SFM. The NS should be thawed in a 37° C. water bath and swirled, not vortexed or shaken, until it is completely in solution. While swirling, it should be noted whether there are any lipids that are not yet in solution. If lipids are present and the NS is not uniform in appearance, NS is returned to the water bath and the swirling process repeated until it is uniform in appearance. Sometimes this component goes into solution immediately, sometimes after a couple of swirling cycles, and sometimes not at all. If, after a couple of hours, the NS is still not in solution, it is discarded and a fresh unit is used. NS that appears non-uniform after thaw should not be used.

Expansion of CD34+ Cells

A starting population of CD34-positive (CD34+) cells of relatively small number (1-5×10⁶ cells) was expanded to a relatively large number of progenitor cells (about 2-4×10⁹ cells) using the culture media and methods described below. The CD34+ cells (from a single donor) were obtained from Allcells (Berkeley, Calif.). Because there is a degree of variation in the quality and number of CD34+ cells that Allcells typically provides, the newly delivered cells were transferred to a 15 mL conical tube and brought up to 10 mL in CM prior to use.

On day 0, a cell count was performed on the viable (phase-bright) cells and the cells were spun at 1200 rpm to pellet. The cells were resuspended to a density of 275,000 cells/mL with CM containing 200 ng/nL recombinant human Stem Cell Factor (“SCF”; Peprotech, Catalog No. 300-07) and 20 ng/mL human flt-3 ligand (Peprotech, Catalog No. 300-19) (“CM/SCF/flt-3 medium”). On about day 4 or 5, the density of the culture was checked by performing a cell count and the culture was diluted to a density of 275,000 cells/mL with fresh CM/SCF/flt-3 medium. On about day 7, the culture was transferred to a sterile tube and a cell count was performed. The cells were spun at 1200 rpm and resuspended to a density of 275,000 cells/nL with fresh CM/SCF/flt-3 medium.

This cycle was repeated, starting from day 0, a total of 3-5 times over the expansion period.

When the culture is large and being maintained in multiple flasks and is to be resuspended, the contents of all of the flasks are combined into a single container prior to performing a cell count. This ensures that an accurate cell count is achieved and provides for a degree of uniformity of treatment for the entire population. Each flask is checked separately for contamination under the microscope prior to combining to prevent contamination of the entire population.

Between days 17-24, the culture can begin to go into decline (i.e., approximately 5-10% of the total number of cells die) and fail to expand as rapidly as before. The cells are then monitored on a daily basis during this time, as complete failure of the culture can take place in as short as 24 hours. Once the decline has begun, the cells are counted, spun down at 850 rpm for 15 minutes, and resuspended at a density of 350,000 cells/mL in CM/SCF/flt-3 medium to induce one or two more divisions out of the culture. The cells are monitored daily to avoid failure of the culture.

When greater than 15% cell death is evident in the progenitor cell culture and some debris is present in the culture, the progenitor cells are ready to be differentiated.

Differentiation of Progenitor Cells into Mucosal Mast Cells

A second phase is performed to convert the expanded progenitor cells into differentiated mucosal mast cells. These mucosal cultured human mast cells (“CHMC”) are derived from CD34+ cells isolated from umbilical cord blood and treated to form a proliferated population of progenitor cells, as described above. To produce the progenitor cells, the resuspension cycle for the culture was the same as that described above, except that the culture was seeded at a density of 425,000 cells/mL and 15% additional media was added on about day four or five without performing a cell count. Also, the cytokine composition of the medium was modified such that it contained SCF (200 ng/mL) and recombinant human IL-6 (200 ng/mL; Peprotech, Catalog No. 200-06 reconstituted to 100 ug/mL in sterile 10 mM acetic acid) (“CM/SCF/IL-6 medium”).

Phases I and II together span approximately 5 weeks. Some death and debris in the culture is evident during weeks 1-3 and there is a period during weeks 2-5 during which a small percentage of the culture is no longer in suspension, but is instead attached to the surface of the culture vessel.

As during Phase I, when the culture is to be resuspended on day seven of each cycle, the contents of all flasks are combined into a single container prior to performing a cell count to ensure uniformity of the entire population. Each flask is checked separately for contamination under the microscope prior to combining to prevent contamination of the entire population.

When the flasks are combined, approximately 75% of the volume is transferred to the communal container, leaving behind about 10 mL or so in the flask. The flask containing the remaining volume was rapped sharply and laterally to dislodge the attached cells. The rapping was repeated at a right angle to the first rap to completely dislodge the cells.

The flask was leaned at a 45 degree angle for a couple of minutes before the remaining volume was transferred to the counting vessel. The cells were spun at 950 rpm for 15 min prior to seeding at 35-50 mL per flask (at a density of 425,000 cells/mL).

Differentiation of Progenitor Cells into Connective Tissue-Type Mast Cells

A proliferated population of progenitor cells is prepared as above and treated to form a tryptase/chymase positive (connective tissue) phenotype. The methods are performed as described above for mucosal mast cells, but with the substitution of IL-4 for IL-6 in the culture medium. The cells obtained are typical of connective tissue mast cells.

Differentiation of Progenitor Cells into Basophil Cells

A proliferated population of progenitor cells is prepared as described above, and used to form a proliferated population of basophil cells. The progenitor cells are treated as described for mucosal mast cells, but with the substitution of IL-3 (at 20-50 ng/mL) for IL-6 in the culture medium.

### Example 2

### Priming and Stimulation Conditions for FcγRI Activation of Cultured Human Mast Cells (CHMC)

The culture media contained the following components:
a) Gibco's StemPro-34 SFM Complete medium;
   StemPro-34 SFM, Catalog No. 10640, 500 mL
   StemPro-34 Nutrient Supplement, Catalog No. 10641, 13 mL
b) L-Glutamine: 200 mM Solution, Mediatech, Catalog No. MT 25-005-CI
   add 5 mL per 500 mL StemPro
c) Penicillin/Streptomycin Soln. 100×, HyClone, Catalog No. SV30010
   add 5 mL per 500 mL StemPro.

Priming of mast cells is carried out by seeding the cells on Day 0 at 3.5×10⁵ cells/mL in culture media plus SCF (200 ng/mL), IL-6 (200 ng/mL), IL-4 (20 ng/nL), IgE (200 ng/mL), and IgG3 (200 ng/mL).

Activation or stimulation of cells is done on Day 3 by centrifuging the cells down and resupending them at 1.5×10³ cells/mL in modified tyrodes (MT: 137 mM NaCl, 2.7 mM KCI, 1.8 mM CaCl₂, 5.6 mM glucose, 20 mM Hepes (pH 7.4), 0.1% Bovine Serum Albumin (Sigma A4503)), with or without rabbit anti-human IgG (200 ng/mL final conc.), for 30 minutes at 37° C. (see Demo, S. D., Cytometry. 36(4):340-348 (1999)).

Degranulation is measured by centrifuging down the sample, and harvesting the supernatant and measuring tryptase activity. The tryptase assays are performed under standard conditions (25 uL supernatant added to 100 uL of 10 uM tryptase substrate [20 mM stock solution of Z-Ala-Lys-Arg-AMC2TFA (Enzyme systems Products, Catalog No. AMC-246) diluted 1:2000 in tryptase assay buffer [0.1 MM Hepes (pH 7.5), 10% w/v Glycerol, 10 eM Heparin, 0.01% NaN₃] for 30 min at 37° C. The reactions are read in plate reader (Wallac, Victor 2, 1420 Multilabel Counter).

### Example 3

### Screening of Small Molecule Candidate Compounds for Modulation of CHMC IgE and/or IgG Activation

To duplicate 96-well U-bottom plates (Costar 3799) are added 65 ul of compound dilutions or control samples prepared in MT [137 mM NaCl,2.7 mM KCL 1.8 MM CaCl₂, 5.6 mM glucose, 20 mM Hepes (pH 7.4), 0.1% Bovine Serum Albumin (Sigma A4503)] containing 2% MeOH and 1% DMSO, and primed with IgE as in Example 2. The mast cell modulating compounds comprise 2,4-pyrimidinediamine compounds disclosed in U.S. patent application Ser. No. 10/355,543, hereby incorporated by reference. CMHC cells are pelleted (980 rpm, 10 min) and resuspended in warm MT. To each 96-well plate is added 65 ul of cells (1000-1500 cells/well). After mixing four times, the cells are incubated for 1 hr at 37° C. and then stimulated with anti-IgE and/or anti-IgG. For controls, MT is added to wells containing unstimulated cells. Stimulation is for 30 minutes at 37° C. Assay for tryptase activity is carried out as above. Leukotriene C4 in the supernatant is quantified using ELISA kit (Cayman Chemical Co., Ann Arbor, Mich.; Catalog No. 520211) following supplier's instructions.

What is claimed:

1. A method of identifying a compound capable of modulating Fcγ receptor signaling pathway, comprising
a) contacting at least one IgE primed mast cell with a candidate compound in the presence of Fcγ receptor signaling activation; and
b) determining whether the candidate compound modulates the Fcγ receptor-mediated signaling cascade.

2. The method of claim 1 in which the candidate compound inhibits the Fcγ receptor signaling cascade.

3. The method of claim 1 in which the Fcγ receptor is FcγRI.

4. The method of claim 1 in which the mast cell is a cultured mast cell.

5. The method of claim 1 in which the mast cell is a mucosal mast cell.

6. The method of claim 1 in which the mast cell is a human mast cell.

7. The method of claim 1 in which the modulation of Fcγ receptor signaling cascade is determined by measuring degranulation.

8. The method of claim 7 in which the modulation of Fcγ receptor signaling is determined by comparing Fcγ receptor-mediated mast cell activation in presence and absence of the candidate compound.

9. The method of claim 1 in which the compound is a small organic compound.

10. The method of claim 9 in which the small organic compound has a molecular weight in the range of about 100-2500 daltons.

11. A method of identifying a compound for treating disorders of IgG-mediated mast cell activation, comprising:
a) contacting at least one IgE primed mast cell with a candidate compound in the presence of Fcγ receptor signaling activation; and
b) determining whether the candidate compound modulates the Fcγ receptor signaling cascade.

12. The method of claim 11 in which the candidate compound inhibits the Fcγ receptor signaling cascade.

13. The method of claim 11 in which the Fcγ receptor is FcγRI.

14. The method of claim 11 in which the mast cell is a cultured mast cell.

15. The method of claim 11 in which the mast cell is a mucosal mast cell.

16. The method of claim 11 in which the mast cell is a human mast cell.

17. The method of claim 11 in which modulation of the Fcγ receptor signaling cascade is determined by measuring degranulation.

18. The method of claim 11 in which modulation of the Fcγ receptor signaling cascade is determined by comparing Fcγ receptor-mediated mast cell activation in presence and absence of the candidate compound.

19. The method of claim 11 in which the compound is a small organic compound.

20. The method of claim 19 in which the small organic compound has a molecular weight in the range of about 100-2500 daltons.

21. A method of identifying a compound capable of modulating IgE priming of mast cells, comprising:
a) contacting at least one mast cell with a candidate compound and priming the mast cell with IgE antibody,
b) activating signal transduction via Fcγ receptor-mediated signaling pathway, and
c) determining whether the candidate compound modulates IgE priming of the mast cell.

22. The method of claim 21 in which the candidate compound inhibits IgE priming of the mast cell.

23. The method of claim 21 in which the Fcγ receptor is FcγRI.

24. The method of claim 21 in which the mast cell is a cultured mast cell.

25. The method of claim 21 in which the mast cell is a mucosal mast cell.

26. The method of claim 21 in which the mast cell is a human mast cell.

27. The method of claim 21 in which modulation of the IgE priming of the mast cell is determined by measuring degranulation.

28. The method of claim 21 in which modulation of the IgE priming of mast cell is determined by comparing IgE priming in presence and absence of the candidate compound.

29. The method of claim 21 in which the compound is a small organic compound.

30. The method of claim 29 in which the small organic compound has a molecular weight in the range of about 100-2500 daltons.
